(19) United States
(12) Patent Application Publication
(10) Pub. No.: US 2005/0102006 A1
(43) Pub. Date: May 12, 2005

(54) Skull-mounted electrical stimulation system

(76) Inventors: Todd K. Whitehurst, Santa Clarita, CA (US); Rafael Carbunaru, Studio City, CA (US); Albert A. Maltan, Sistrans (AT); Michael A. Faltys, Northridge, CA (US)

Correspondence Address: ADVANCED BIONICS CORPORATION, 25129 RYE CANYON ROAD, VALENCIA, CA, 91355, US

(21) Appl. No.: 10/949,067

(22) Filed: Sep. 24, 2004

Related U.S. Application Data
(60) Provisional application No. 60/505,831, filed on Sep. 25, 2003.

(51) Int. Cl. A61N 1/18

(52) U.S. Cl. 607/46

(57) ABSTRACT

Systems and methods for applying electrical stimulation to the brain to treat headaches and neuralgia use at least one implantable system control unit (SCU), specifically an implantable signal/pulse generator (IPG) with one or more electrodes. The IPG is implanted in the skull and communicates with at least one external appliance, such as a Behind-the-Ear (BTE) unit. In a preferred embodiment, the system is capable of open- and closed-loop operation. In closed-loop operation, at least one SCU includes a sensor, and the sensed condition is used to adjust stimulation parameters.

The present application claims the benefit of U.S. Provisional Patent Application Ser. No. 60/505,831, filed Sep. 25, 2003, which application is incorporated herein by reference in its entirety. The present application is also related to U.S. patent application Ser. No. 10/934,155, filed Sep. 3, 2004, and U.S. Pat. No. 6,735,475, issued May 11, 2004, both of which documents are incorporated herein by reference in their entireties.

### FIELD OF THE INVENTION

The present invention generally relates to implantable stimulator systems and methods, and more particularly relates to implantable stimulator systems and methods utilizing one or more implantable leads for treating headache and/or occipital neuralgia.

### BACKGROUND OF THE INVENTION

The public health significance of headache pain and occipital neuralgia is often overlooked, probably because of their episodic nature and the lack of mortality attributed to them. Headache disorders and occipital neuralgia are, however, often incapacitating, with considerable impact on social activities and work, and may lead to significant consumption of drugs with adverse side effects.

The International Headache Society (IHS) published “Classification and Diagnostic Criteria for Headache Disorders, Cranial Neuralgias and Facial Pain” in 1988. IHS identified 13 different general groupings of headache, given below in Table 1.

TABLE 1
Groupings of Headache Disorders and Facial Pain

```
  1. Migraine
  2. Tension-type headache
  3. Cluster headache and chronic paroxysmal hemicrania
  4. Miscellaneous headaches unassociated with structural lesions
  5. Headache associated with head trauma
  6. Headache associated with vascular disorders
  7. Headache associated with non-vascular intracranial disorder
  8. Headache associated with substances or their withdrawal
  9. Headache associated with non-cephalic infections
10. Headaches associated with metabolic disorders
11. Headache or facial pain associated with disorder of cranium,
  neck, eyes, ears, nose, sinuses, teeth, mouth or other facial or
  cranial structures
12. Cranial neuralgias, nerve trunk pain and deafferentation pain
13. Non-classifiable headache
```

The IHS classification of the most common types of headache is summarized in Table 2, below.

TABLE 2
IHS Classification of Primary Headaches

```
1. Migraine
  1.1 Migraine without aura
  1.2 Migraine with aura
    1.2.1 Migraine with typical aura
    1.2.2 Migraine with prolonged aura
    1.2.3 Familial hemiplegic migraine headache
    1.2.4 Basilar migraine
    1.2.5 Migraine aura without headache
    1.2.6 Migraine with acute onset aura
  1.3 Ophthalmoplegic migraine
  1.4 Retinal migraine
  1.5 Childhood periodic syndromes that may be precursors to or
    associated with migraine
    1.5.1 Benign paroxysmal vertigo of childhood
    1.5.2 Alternating hemiplegia of childhood
  1.6 Complications of migraine
    1.6.1 Status migrainosus
    1.6.2 Migrainous infarction
  1.7 Migrainous disorder not fulfilling above criteria
2. Tension-type headache
  2.1 Episodic tension-type headache
    2.1.1 Episodic tension-type headache associated with disorder of
        pericranial muscles
    2.1.2 Episodic tension-type headache not associated with disorder
        of pericranial muscles
  2.2 Chronic tension-type headache
    2.2.1 Chronic tension-type headache associated with disorder of
        pericranial muscles
    2.2.2 Chronic tension-type headache not associated with disorder
        of pericranial muscles
  2.3 Headache of the tension-type not fulfilling above criteria
3. Cluster headache and chronic paroxysmal hemicrania
  3.1 Cluster Headache
    3.1.1 Cluster headache, periodicity undetermined
    3.1.2 Episodic cluster headache
    3.1.3. Chronic Cluster Headache
    3.1.3.1 Unremitting from onset
    3.1.3.2 Evolved from episodic
  3.2 Chronic paroxysmal hemicrania
  3.3 Cluster headache-like disorder not fulfilling above Criteria
```

Migraine Headache

The IHS classification provides diagnostic criteria for migraine without and with aura, summarized in Tables 3 and 4 below.

TABLE 3
IHS Diagnostic Criteria for Migraine Without Aura

```
A. At least five attacks fulfilling B-D
B. Headache attacks lasting 4-72 h (untreated or unsuccessfully treated)
C. Headache has at least two of the following characteristics:
  1. Unilateral location
  2. Pulsating quality
  3. Moderate or severe intensity (inhibits or prohibits daily activities)
  4. Aggravation by walking stairs or similar routine physical activity
D. During headache at least one of the following:
  1. Nausea and/or vomiting
  2. Photophobia and phonophobia
E. At least one of the following:
  1. History and physical do not suggest headaches secondary to organic
    or systemic metabolic disease
  2. History and/or physical and/or neurologic examinations do suggest
    such disorder, but is ruled out by appropriate investigations
  3. Such disorder is present, but migraine attacks do not occur for the
    first time in close temporal relation to the disorder
```

TABLE 4
IHS Diagnostic Criteria for Migraine With Aura

```
A. At least two attacks fulfilling B
B. At least three of the following four characteristics:
  1. One or more fully reversible aura symptoms indicating focal cerebral
    cortical and/or brain stem dysfunction
  2. At least one aura symptom develops gradually over more than four
    minutes or two or more symptoms occur in succession
  3. No aura symptom lasts more than 60 minutes. If more than one aura
    symptom is present, accepted duration is proportionally increased
  4. Headache follows aura with a free interval of less than 60 minutes. It
    may also begin before or simultaneously with the aura.
C. At least one of the following:
  1. History and physical and neurologic examinations do not suggest
    headaches secondary to organic or systemic metabolic disease
  2. History and/or physical and/or neurologic examinations do suggest
    such disorder, but it is ruled out by appropriate investigations
  3. Such disorder is present, but migraine attacks do not occur for the
    first time in close temporal relation to the disorder
```

The IHS classification includes several different types of migraine variants. Basilar migraine is defined as a migraine with an aura involving the brainstem. Symptoms include ataxia, dysarthria, vertigo, tinnitus and/or changes in consciousness and cognition. Ophthalmoplegic migraine is associated with acute attacks of third nerve palsy with accompanying dilation of the pupil. In this setting, the differential diagnosis includes an intracranial aneurysm or chronic sinusitis complicated by a mucocele. The ophthalmoplegia can last from hours to months. Hemiplegic migraine is distinguished by the accompanying hemiplegia, which can be part of the aura, or the headache may precede the onset of hemiplegia. Hemiplegic migraine can be familial and may last for days or weeks, clinically simulating a stroke. An additional differential diagnosis includes focal seizures.

Status migrainosus describes a migraine lasting longer than 72 hours with intractable debilitating pain, and typically occurs in a setting of inappropriate and prolonged use of abortive anti-migraine drugs. These patients may require hospitalization, both for pain control, detoxification from the abused drugs, and treatment of dehydration resulting from prolonged nausea and vomiting.

A migraine prevalence survey of American households was conducted in 1992, and included 20,468 respondents 12-80 years of age. Using a self-administered questionnaire based on modified IHS criteria, 17.6% of females and 5.7% of males were found to have one or more migraine headaches per year. A projection to the total US population suggests that 8.7 million females and 2.6 million males suffer from migraine headache with moderate to severe disability. Of these, 3.4 million females and 1.1 million males experience one or more attacks per month. Prevalence is highest between the ages of 25 and 55, during the peak productive years.

Based on published data, the Baltimore County Migraine Study, MEDSTAT's MarketScan medical claims data set, and statistics from the Census Bureau and the Bureau of Labor Statistics, it has been estimated that migraineurs require 3.8 bed rest days for men and 5.6 days for women each year, resulting in a total of 112 million bedridden days. Migraine costs American employers about $13 billion a year because of missed workdays and impaired work function; close to $8 billion is directly due to missed workdays. Patients of both sexes aged 30 to 49 years incurred higher indirect costs compared with younger or older employed patients. Annual direct medical costs for migraine care are about $1 billion, with about $100 spent per diagnosed patient. Physician office visits account for about 60% of all costs; in contrast, emergency department visits contribute less than 1% of the direct costs.

Tension-Type Headache

The diagnostic criteria for tension-type headaches are summarized in Table 5, below. However, migraine symptoms may overlap considerably with that of tension-type headaches. Tension-type headaches are believed by some experts to be a mild variant of migraine headache. Patients with tension-type headaches who also have migraines may experience nausea and vomiting with a tension headache, though when they do, it typically is mild and for a shorter duration compared to that with a migraine. Tension-type headache may be a disorder unto itself in individuals who do not have migraines, and may manifest as attacks of mild migraine in individuals with migraines.

TABLE 5
IHS Criteria for Various Forms of Tension-Type Headache

```
Tension-type headache
At least two of the following pain characteristics:
1. Pressing/tightening (non-pulsating) quality
2. Mild or moderate intensity (may inhibit, but does not prohibit
  activities)
3. Bilateral location
4. No aggravation by walking stairs or similar routine physical activity
Both of the following:
1. No nausea or vomiting (anorexia may occur)
2. Photophobia and phonophobia absent, or only one is present
At least one of the following:
1. History and physical do not suggest headaches secondary to organic
  or systemic metabolic disease
2. History and/or physical and/or neurologic examinations do suggest
  such disorder, but is ruled out by appropriate investigations
3. Such disorder is present, but tension-type headache does not occur for
  the first time in close temporal relation to the disorder
Episodic tension-type headache (ETTH)
Diagnostic criteria:
A. At least 10 previous episodes, <180 days/year (<15/mo) with
headache
B. Headache lasting from 30 minutes to 7 days
Chronic tension-type headache (CTTH)
Diagnostic criteria:
A. Average frequency ≧1 day/month (≧189 days/year) for ≧6 months
Tension-type headache associated with
disorder of pericranial muscles
At least one of the following:
1. Increased tenderness of pericranial muscles demonstrated by manual
  palpation or pressure algometer.
2. Increased electromyographic level of pericranial muscles at rest or
  during physiologic tests.
Tension-type headache not associated with
pericranial muscle disorder
No increased tenderness of pericranial muscles. If studied,
electromyography of pericranial muscles shows normal levels
of activity.
```

Based on a telephone survey of 13,345 people, the 1-year period prevalence of episodic tension-type headache (ETTH) is estimated to be 38.3%, according to IHS criteria. Women had a higher 1-year ETTH prevalence than men in all age, race, and education groups, with an overall prevalence ratio of 1.16. Prevalence peaked in the 30- to 39-year-old age group in both men (42.3%) and women (46.9%). Prevalence increased with increasing educational levels in both sexes, reaching a peak in subjects with graduate school educations of 48.5% for men and 48.9% for women. Of subjects with ETTH, 8.3% reported lost workdays because of their headaches, while 43.6% reported decreased effectiveness at work, home, or school.

Chronic Daily Headache

Chronic tension-type headache (CTTH) is a subtype of tension headaches, with patients experiencing headaches daily or almost every day. In practice, the term “chronic daily headache” is commonly used to describe headaches lasting for greater than 4 hours per day and for at least 15 days per month. The classification of chronic daily headaches is summarized below in Table 6.

TABLE 6
Classification of Chronic Daily Headache

Transformed migraine
1. With medication overuse
2. Without medication overuse
Chronic tension-type headache (CTTH)
1. With medication overuse
2. Without medication overuse
New daily persistent headache
1. With medication overuse
2. Without medication overuse
Hemicrania continua
1. With medication overuse
2. Without medication overuse

In the study of 13,345 people cited above, the 1-year period prevalence of chronic tension-type headache (CTTH) was estimated to be 2.2%. This prevalence was higher in women and declined with increasing education. Subjects with CTTH reported more lost workdays (mean of 27.4 days vs. 8.9 days for those reporting lost workdays) and reduced-effectiveness days (mean of 20.4 vs. 5.0 days for those reporting reduced effectiveness) compared with subjects with ETTH.

Chronic daily headaches are best conceptualized as an umbrella category term, referring to a group of headache disorders characterized by headaches which occur greater than 15 days per month, with an average untreated duration of greater than 4 hours per day. There are many secondary causes of chronic daily headache, including post-traumatic headache, arteritis, intracranial mass lesions, etc. There are also short-lived primary headache disorders that occur greater than 15 days per month, such as chronic cluster headache or the paroxysmal hemicranias. These secondary and short-lived disorders are outside the scope of this discussion. The most common primary, chronic daily headache disorders include transformed migraine, chronic tension-type headaches, new daily persistent headache, or hemicrania continua. Each of these diagnoses can be complicated by medication overuse (e.g., barbiturates, acetaminophen, aspirin, caffeine, ergotamine tartrate and opioids). When used daily, all of these medications can lead to a vicious cycle of rebound headaches.

Cluster Headache

The 1988 IHS classification system recognized the uniqueness of cluster headache as a clinical and epidemiological entity. Formerly classified as a vascular migraine variant, cluster headache (a.k.a. suicide headache) is thought to be one of the most severe headache syndromes. It is characterized by attacks of severe pain, generally unilateral and orbital and lasting 15 minutes to 3 hours, with one or more symptoms such as unilateral rhinorrhea, nasal congestion, lacrimation, and conjunctival injection. In most patients, headaches occur in episodes, generally with a regular time pattern. These “cluster periods” last for weeks to months, separated by periods of remission lasting months to years. It primarily affects men, and in many cases, patients have distinguishing facial, body, and psychological features. Several factors may precipitate cluster headaches, including histamine, nitroglycerin, alcohol, transition from rapid eye movement (REM) to non-REM sleep, circadian periodicity, environmental alterations, and change in the level of physical, emotional, or mental activity. The IHS classification system gives specific diagnostic criteria for cluster headache, as given in Table 7 below.

TABLE 7
IHS Diagnostic Criteria for Cluster Headache

3.1 Cluster Headache

| A. | At least 5 attacks fulfilling B-D | |
|---|---|---|
| B. | Severe unilateral, orbital, supraorbital and/or temporal pain lasting 15-180 | |
| | minutes untreated | |
| C. | At least one of the following signs present on the pain side: | |
| | 1. | Conjunctival injection |
| | 2. | Lacrimation |
| | 3. | Nasal congestion |
| | 4. | Rhinorrhea |
| | 5. | Forehead and facial sweating |
| | 6. | Miosis |
| | 7. | Ptosis |
| | 8. | Eyelid edema |
| D. | Frequency of attacks: from 1 every other day to 8 per day | |
| E. | At least one of the following: | |
| | 1. | History, physical and neurological examinations do not suggest one of the |
| | | disorders listed in groups 5-11 of Table 1 |
| | 2. | History and/or physical and/or neurological examinations do suggest such |
| | | disorder, but it is ruled out by appropriate investigations |
| | 3. | Such disorder is present, but cluster headache does not occur for the first |
| | | time in close temporal relation to the disorder |

3.1.1 Cluster headache periodicity undefined

| | |
|---|---|
| A. | Criteria for 3.1 fulfilled |
| B. | Too early to classify as 3.1.2 or 3.1.3 |

3.1.2 Episodic cluster headache
Description: Attacks lasting between 1 week and 3 months occur in periods
lasting 1 week to one year separated by pain free periods lasting 14 days
or more.

| | |
|---|---|
| A. | All the letter headings of 3.1 |
| B. | At least 2 periods of headaches (cluster periods) lasting (untreated) from 7 |
| | days to one year, separated by remissions of at least 14 days. |

3.1.3 Chronic cluster headache
Description: Attacks lasting between 2 weeks and 3 months occur for more
than one year without remission or with remissions lasting less than 14
days.

| | |
|---|---|
| A. | All the letter headings of 3.1 |
| B. | Absence of remission phases for one year or more or with remissions |
| | lasting less than 14 days. |

3.1.3.1 Chronic cluster headache unremitting from onset

| | |
|---|---|
| A. | All the letter headings of 3.1.3 |
| B. | Absence of remission periods lasting 14 days or more from onset. |

3.1.3.2 Chronic cluster headache evolved from episodic

| | |
|---|---|
| A. | All the letter headings of 3.1.3 |
| B. | At least one interim remission period lasting 14 days or more within one |
| | year after onset, followed by unremitting course for at least one year. |

The estimated prevalence of cluster headache is 69 cases per 100,000 people. Men are affected more commonly than women in a proportion of 6:1. Although most patients begin experiencing headache between the ages of 20 and 50 years (mean of 30 years), the syndrome may begin as early as the first decade and as late as the eighth decade.

Cervicogenic Headache

Cervicogenic headache (CEH) is a headache with its origin in the neck area. The source of pain is in structures around the neck that have been damaged. These structures can include joints, ligaments, muscles, and cervical discs, all of which have complex nerve endings. When these structures are damaged, the nerve endings send pain signals up the pathway from the upper nerves of the neck to the brainstem. These nerve fibers may synapse in the same brainstem nuclei as the nerve fibers of the trigeminal nerve. Since the trigeminal nerve is responsible for the perception of head pain, the patient experiences the symptoms of headache and/or facial pain.

While many patients who are diagnosed with CEH have the traditional symptoms of tension-type headache, some of the patients who have the traditional symptoms of migraine and cluster headache also respond to CEH diagnosis and treatment.

Occipital Neuralgia

Occipital neuralgia is a chronic pain disorder caused by irritation or injury to the occipital nerves of the suboccipital region and the back of the head. Occipital neuralgia causes significant pain, characterized by a continuous throbbing or migraine-like aching which originates in the neck and spreads up and around the forehead and scalp. Causes of occipital neuralgia include physical stress, trauma to or compression of the greater or lesser occipital nerves, tumors involving the second and third cervical dorsal roots, or repeated contraction of the muscles of the neck. Current treatment of severe cases of occipital neuralgia includes prescribing antidepressant drugs or injecting steroids into affected areas.

Treatment Using a Microstimulator

To treat migraine, tension-type headache, cluster headache, cervicogenic headache, other types of headache, and/or facial pain, the use of a miniature implantable neurostimulator (also referred to as a BION® device and/or microstimulator) has been suggested. A BION microstimulator may be implanted via a minimal surgical procedure (e.g., injection or small incision) adjacent to any nerve(s) arising from the upper cervical spine (i.e., C1-C4), including a greater occipital nerve(s), a lesser occipital nerve(s), a third occipital nerve(s), a great auricular nerve(s), a transverse cervical nerve(s), a supraclavicular nerve(s), or a branch(es) of any of these neural structures to treat migraine, tension-type headache, cluster headache, cervicogenic headache, other types of headache, and occipital neuralgias.

However, BION devices and other microstimulators have limited battery supply and are difficult to explant due to their relatively small size. Yet, some patients treated for migraines, tension-type headaches, cluster headaches, cervicogenic headaches, other types of headaches, and occipital neuralgias require continuous stimulation at a frequency of at least 50-100 Hz. Such a high frequency of stimulation is likely to quickly deplete the microstimulators' battery supplies and thus require frequent recharging and consequent explantation of the microstimulators within a relatively short period of time, i.e., about three years.

### BRIEF SUMMARY OF THE INVENTION

The present invention provides means for chronically stimulating any nerve(s) arising from the upper cervical spine (i.e., C1-C4), including: a greater occipital nerve(s), a lesser occipital nerve(s), a third occipital nerve(s), a great auricular nerve(s), a transverse cervical nerve(s), a supraclavicular nerve(s), or a branch(es) of any of these neural structures with an implantable neurostimulator. The present invention provides systems and methods for applying electrical stimulation to one or more nerves arising from these nerves via a “skull-mounted” device. Electrical stimulation of such targets may provide significant therapeutic benefit in the management of migraine, tension-type headache, cluster headache, cervicogenic headache, other types of headache, and/or occipital neuralgia. Such therapeutic benefit may be provided through “field stimulation”, or stimulation directly to, on, or in the area where the pain is perceived. Alternatively and/or additionally, therapeutic benefit may be provided through stimulation to a nerve or tissue that is remote from, or located in a different area than, the area where the pain is perceived.

The treatment provided by the invention is carried out by employing at least one system control unit (SCU). In one preferred form, and SCU comprises an implantable pulse generator (IPG), and external Behind-the-Ear (BTE) unit, and implantable electrode(s). In this embodiment, the SCU is preferably implanted in a surgically-created shallow depression in the skull (e.g., the mastoid area), with one or more electrode leads attached to the SCU extending subcutaneously towards the nerve(s) arising from the upper cervical spine. Preferred systems also include one or more sensors for sensing symptoms or other conditions that may indicate a need for treatment.

The IPG includes a battery that is much larger than a battery of a typical microstimulator, thus extending the time between recharges and consequent explantation procedures which may be expensive, time consuming, and potentially uncomfortable for patients. The BTE unit is adapted be situated on the exterior of a patient, near the location where the IPG is imbedded within the skull (e.g., the mastoid bone). The BTE unit includes circuitry and an coil used to recharge the IPG transcutaneously.

The SCU preferably includes a programmable memory for storing data and/or control stimulation parameters. This allows stimulation and control parameters to be adjusted to levels that are safe and efficacious with minimal discomfort. Electrical stimulation may be controlled independent of any other stimulation or drug infusion system; alternatively, the SCU may be combined to operate with other electrical and drug stimulation systems to provide various therapy to a patient.

According to a preferred embodiment of the invention, the electrodes used for electrical stimulation are arranged as an array on a very thin implantable lead. The SCU is programmed to produce either monopolar electrical stimulation, e.g., using the SCU case as an indifferent electrode, or to produce bipolar and/or multipolar electrical stimulation, e.g., using one or more of the electrodes of an electrode array as an indifferent electrode. The SCU includes a means of stimulating (a) nerve(s) either intermittently or continuously. Specific stimulation parameters may provide therapeutic advantages for, e.g., various forms of headaches or neuralgia.

An exemplary, but not limiting, SCU used with the present invention preferably possesses one or more of the following properties:
at least one electrode for applying stimulating current to surrounding tissue;
electronic and/or mechanical components encapsulated in a hermetic package made from biocompatible material(s);
an electrical coil inside the package that receives power and/or data by inductive or radio-frequency (RF) coupling with a transmitting coil placed outside the body in a BTE unit (or any other head-mounted unit), avoiding the need for electrical leads to connect devices to a central implanted or external controller;
a coil or transmitter for receiving and/or transmitting signals via telemetry;
means for receiving and/or storing electrical power within the SCU; and
a form factor making the SCU implantable in a depression or opening cut in, e.g., the mastoid area of the skull.

The power source of the SCU is preferably realized using one or more of the following options, or means for providing operating power:
(1) an external BTE power source coupled to the SCU via an RF link;
(2) a self-contained power source made using any means of generation or storage of energy, e.g., a primary battery, a replenishable or rechargeable battery, a capacitor, a supercapacitor; and/or
(3) if the self-contained power source is replenishable or rechargeable, a means of replenishing or recharging the power source, e.g., an RF link, an optical link, or other energy-coupling link.

According to one embodiment of the invention, an SCU operates independently. According to another embodiment of the invention, an SCU operates in a coordinated manner with other implanted SCUs, other implanted devices, or with devices external to the patient's body.

According to yet another embodiment of the invention, an SCU incorporates means of sensing headaches or neuralgia or symptoms thereof, or other measures of the state of the patient. Sensed information is preferably used to control the electrical stimulation parameters of the SCU in a closed-loop manner. According to one embodiment of the invention, the sensing and stimulating means are incorporated into a single SCU. According to another embodiment of the invention, the sensing means communicates sensed information to at least one SCU with stimulating means.

Thus, the present invention provides systems and methods for the treatment of headaches and/or neuralgia that use at least one SCU. The present invention's advantages include, among others: monitoring and programming capabilities; power source, storage, and transfer mechanisms; device activation by the patient or clinician; open- and closed-loop capabilities coupled with sensing a need for and/or response to treatment; simple explantation because the IPG is implanted in the skull (e.g., in the mastoid bone) and all leads are directly attached to the IPG; and coordinated use of one or more SCUs.

### BRIEF DESCRIPTION OF THE DRAWINGS

The above and other aspects of the present invention will be more apparent from the following more particular description thereof, presented in conjunction with the following drawings wherein:

FIG. 1A illustrates the cervical plexus, depicting various nerves and muscles;

FIG. 1B depicts various nerves and muscles of the back of the head and neck;

FIG. 2 illustrates a lateral view of the skull;

FIG. 3 illustrates internal and external components of an embodiment of the invention;

FIG. 4 illustrates external components of an embodiment of the invention; and

FIG. 5 illustrates a Behind-the-Ear (BTE) unit for use with an embodiment of the invention.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

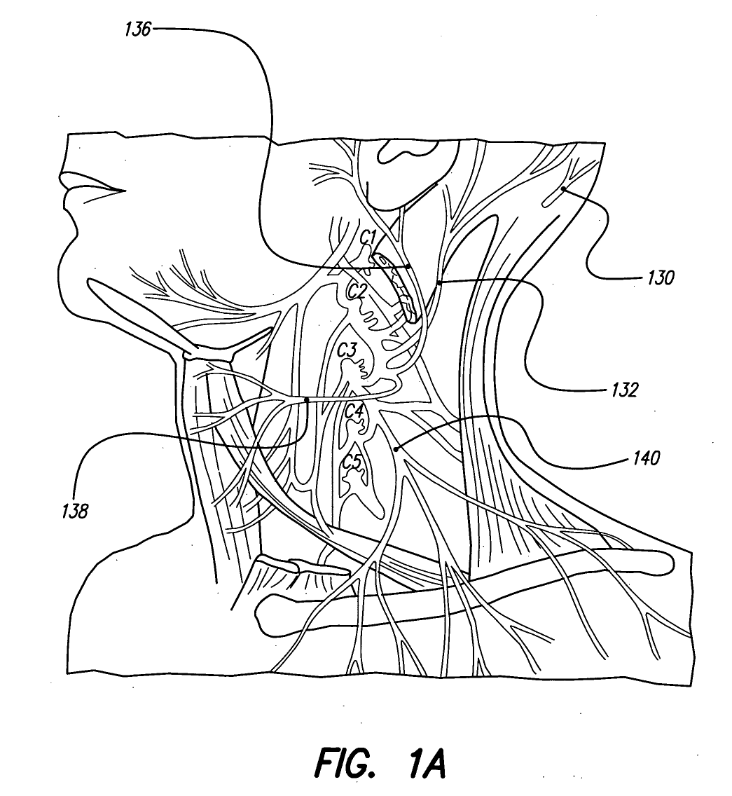

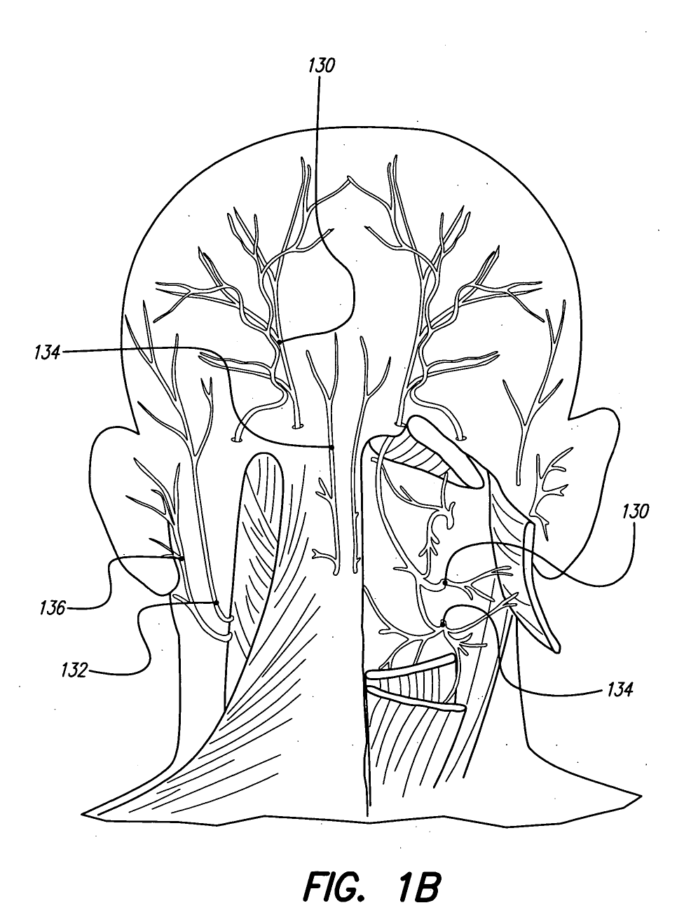

Discussed herein are ways to effectively use such small, fully implantable, chronic neurostimulators for treating headache pain and neuralgia. Headache pain and neuralgia, including occipital neuralgia, may be relieved with stimulation applied to nerves arising from the upper cervical spine (C1-C4). As seen in FIGS. 1A and 1B, many of these nerves are relatively easily accessed, especially in their distal portions, since they lie subcutaneously in the back of the head or the back or sides of the neck. Examples of such nerves are the greater occipital nerve 130, the lesser occipital nerve 132, and the third occipital nerve 134, as well as the great auricular nerve(s) 136, a transverse cervical nerve(s) 138, a supraclavicular nerve(s) 140, and/or branches of any of these. In accordance with additional teachings of the present invention, stimulation of, for instance, one or more of these nerves is alternatively or additionally provided to relieve headache pain and/or neuralgia.

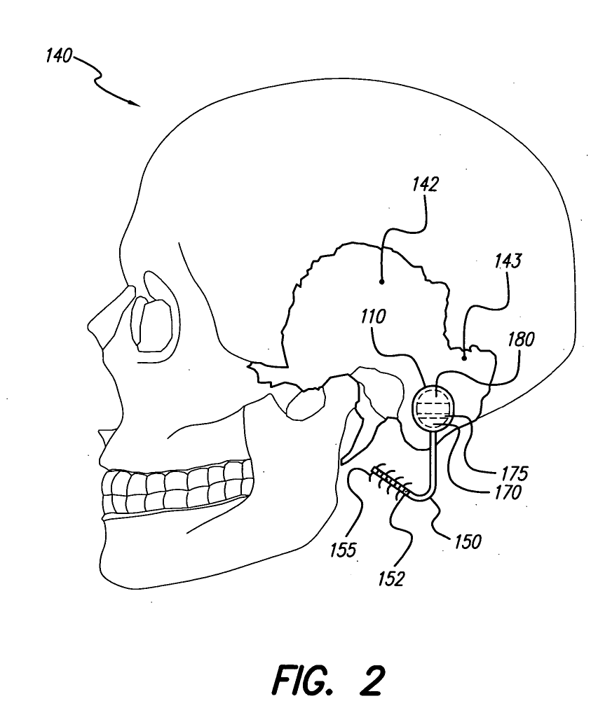

An exemplary embodiment is depicted in FIG. 2 where a system control unit (SCU) 110 is implanted beneath the scalp in a surgically-created shallow depression or opening in the mastoid area 143 of the temporal bone 142 of the skull 140. SCU 110 preferably conforms to the profile of surrounding tissue(s) and/or bone(s), and is small and compact. This is preferable so that no unnecessary pressure is applied to the skin or scalp, as this may result in skin erosion or infection. SCU 110 preferably has a diameter of no greater than 75 mm, more preferably no greater than 65 mm, and most preferably about 35-55 mm. SCU thickness (e.g., depth into the skull) of approximately 10-12 mm is preferred, while a thickness of about 8-10 mm or less is more preferred.

The method of implanting the SCU 110 of the present invention in the mastoid area 143 of the temporal bone 142 of the skull 140 is superior to prior methods of implanting similar devices in the skull for several reasons. First, the method of the present invention contemplates implantation in the mastoid area 143 because the mastoid area 143 of the temporal bone is relatively thicker than other areas of the skull 140. Second, the method of the present invention of implanting SCU 110 in the mastoid area 143 contemplates implantation of the SCU 110 in a shallow recess or depression cut into the skull (e.g., the mastoid area 143) rather than a hole cut entirely through the skull 140. By not cutting a hole through the skull, the method of the present invention maintains maximal integrity of the skull 140 and thereby avoids possible injury and infection that could otherwise accompany an exposure of the fragile tissues of the brain or inner ear. The method of the present invention need not cut a hole through the skull because, as will be shown, lead(s) 150 travel to the upper cervical spine rather than the tissue of the brain. Thus, the present invention is an improvement over prior systems and methods that gracefully avoids unnecessary intracranial intrusions.

One or more electrode lead(s) 150 attached to SCU 110 run subcutaneously, preferably in a surgically-created (a) shallow recess(es) or groove(s) in the mastoid area 143 of the skull 140, to the nerves of the upper cervical spine. Shallowly-recessed placement of the SCU 110 and the lead(s) 150 has the advantages of decreased likelihood of erosion of the overlying skin, and of minimal cosmetic impact. The mastoid area 143 of the temporal bone 142 is a particularly advantageous location to recess the SCU 110 and the lead(s) 150 because the mastoid process is relatively thick in relation to the rest of the bones of the skull, although and SCU of the present invention may be implanted in any other feasible area of the skull.

At least one, and preferably four, electrode(s) 152 are carried on lead(s) 150 having a proximal end coupled to SCU 110. The lead contains wires electrically connecting electrodes 152 to SCU 110. SCU 110 contains electrical components 170 that produce electrical stimulation pulses that travel through the wires of lead(s) 150 and are delivered to electrodes 152, and thus to the tissue of the upper cervical spine that surrounds electrodes 152. To protect the electrical components inside SCU 110, the case of the SCU 110 is preferably hermetically sealed. For additional protection against, e.g. impact, the case is preferably made of metal (e.g. titanium) or ceramic, which materials are also, advantageously, biocompatible. In addition, SCU 110 is preferably Magnetic Resonance Imaging (MRI) compatible, or MRI safe.

Lead(s) 150, and any other leads of the present invention, may include tine(s) 155 (FIG. 2), barbs, suture sleeves, or other anchors or means of anchoring the electrode(s) on such leads in, or near, the nerves of the upper cervical spine. The lead(s) of the present invention are tunneled under the skin to the implantable pulse generator of the SCU 110 where it/they attach to the implantable pulse generator via a connector. A suture sleeve or other fixation device may be placed at any point along the lead(s) to hold it/them in place.

In one embodiment of the present invention, the electrical stimulation may be provided as described in International Patent Application Serial Number PCT/US01/04417 (the '417 application), filed Jan. 12, 2001 (which claims priority to U.S. Provisional Patent Application Ser. No. 60/182,486, filed Feb. 15, 2000), which application is incorporated herein by reference in its entirety. As such, the electrical stimulation of the present invention may be as provided in this PCT application, which is directed to a “Deep Brain Stimulation System for the Treatment of Parkinson's Disease or Other Disorders”.

The present invention may include one or more SCUs to deliver electrical stimulation and/or drug infusion to a patient. These SCUs may include an SCU with an IPG, e.g., as illustrated in FIG. 2; a microstimulator SCU, such as a BION microstimulator; or an SCU with an implantable drug infusion pump. When needed, an SCU provides both electrical stimulation and one or more stimulating drugs. Each of these SCUs may work in communication with each other to provide therapy to a patient in the upper cervical spine.

SCU 110 preferably contains electronic circuitry 170 for receiving data and/or power from outside the body by inductive, radio-frequency (RF), or other electromagnetic coupling. In a preferred embodiment, electronic circuitry 170 includes an inductive coil for receiving and transmitting RF data and/or power, an integrated circuit (IC) chip for decoding and storing stimulation parameters and generating stimulation pulses (either intermittent or continuous), and additional discrete electronic components required to complete the electronic circuit functions, e.g. capacitor(s), resistor(s), coil(s), and the like.

SCU 110 also advantageously includes programmable memory 175 for storing a set(s) of data, stimulation, and control parameters. This feature allows electrical stimulation to be adjusted to settings that are safe and efficacious with minimal discomfort for each individual. Specific parameters may provide therapeutic advantages for various levels and types of headaches and/or neuralgias. For instance, some patients may respond favorably to intermittent stimulation, while others may require continuous treatment for relief. Electrical stimulation parameters are preferably controlled independently. However, in some instances, they are advantageously coupled with the operations of other SCUs, e.g., electrical stimulation of SCU 110 may be programmed to occur only during drug infusion of another SCU.

In addition, parameters, electrode design and number, and overall system configuration may be chosen to target specific neural populations and to exclude others, or to increase neural activity in specific neural populations and to decrease neural activity in others. For example, relatively low frequency neurostimulation (i.e., less than about 50-100 Hz) typically has an excitatory effect on surrounding neural tissue, leading to increased neural activity, whereas relatively high frequency neurostimulation (i.e., greater than about 50-100 Hz) typically has an inhibitory effect, leading to decreased neural activity. The present invention may thus employ electrical stimulation of at least 10 Hz.

The preferred SCU 110 also includes a power source and/or power storage device 180. Possible power options for a stimulation device of the present invention, described in more detail below, include but are not limited to an external power source in a Behind-the-Ear (BTE) unit coupled to the stimulation device, e.g., via: an RF link; a self-contained power source utilizing any means of generation or storage of energy (e.g., a primary battery, a rechargeable battery such as a lithium ion battery, an electrolytic capacitor, or a super- or ultra-capacitor); and, if the self-contained power source is replenishable or rechargeable, means of replenishing or recharging the power source (e.g., an RF link).

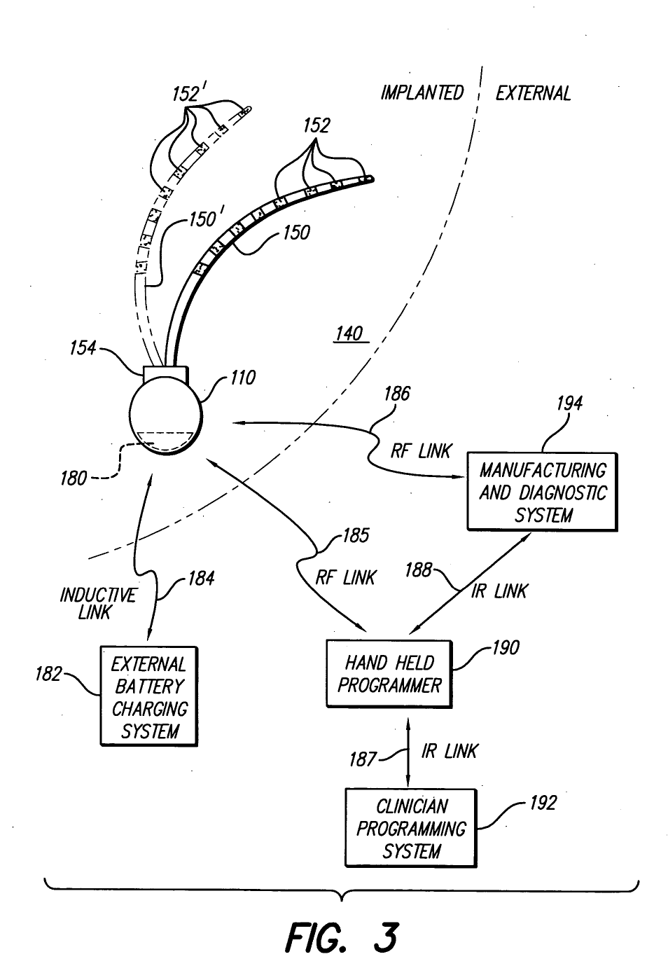

In one embodiment of the present invention shown in FIG. 3, SCU 110 includes a rechargeable battery as a power source/storage device 180. The battery is recharged, as required, from an external battery charging system (EBCS) 182, typically through an inductive link 184. In this embodiment, and as explained more fully in the earlier referenced '417 PCT application, SCU 110 includes a processor and other electronic circuitry 170 that allow it to generate stimulation pulses that are applied to the patient through electrodes 152 in accordance with a program and stimulation parameters stored in programmable memory 175.

According to an embodiment of the present invention, such as described in the previously referenced '417 application and as depicted in FIG. 3, at least one lead 150 is attached to SCU 110, via a suitable connector 154. Each lead includes at least one electrode(s) 152, preferably four electrode(s), and may include as many as sixteen or more electrodes 152. Additional leads 150′ may be attached to SCU 110. Hence, FIG. 3 shows (in phantom lines) a second lead 150′ having electrodes 152′ thereon, also attached to SCU 110.

Lead(s) 150 are preferably less than 5 mm in diameter, and more preferably less than 1.5 mm in diameter. Electrodes 152, 152′ are preferably arranged as an array, more preferably are at least two collinear electrodes, and most preferably at least 4 collinear electrodes. SCU 110 is preferably programmable to produce either monopolar electrical stimulation, e.g., using the SCU case as an indifferent electrode, bipolar electrical stimulation, e.g., using one of the electrodes of the electrode array as an indifferent electrode, or multipolar electrical stimulation. A preferred SCU 110 has at least four channels and drives up to sixteen electrodes or more. Lead(s) 150 may terminate/connect to the SCU 110 using paddle, cylindrical ring, cuff, semi-cuff, or other electrode designs, or any combination thereof.

According to one embodiment of the invention, an SCU operates independently. According to another embodiment of the invention, an SCU operates in a coordinated manner with other SCU(s), other implanted device(s), or other device(s) external to the patient's body. For instance, an SCU may control or operate under the control of another implanted SCU(s), other implanted device(s), or other device(s) external to the patient's body. An SCU may communicate with other implanted SCUs (as mentioned earlier), other implanted devices, and/or devices external to a patient's body via, e.g., an RF link, an ultrasonic link, or an optical link. Specifically, an SCU may communicate with an external remote control (e.g., patient and/or physician programmer) that is capable of sending commands and/or data to an SCU and that is preferably capable of receiving commands and/or data from an SCU.

For example, SCU 110 of the present invention may be activated and deactivated, programmed and tested through a hand held programmer (HHP) 190 (which may also be referred to as a patient programmer and is preferably, but not necessarily, hand held), a clinician programming system (CPS) 192 (which may also be hand held), or a manufacturing and diagnostic system (MDS) 194 (which may also be hand held). HHP 190 may be coupled to SCU 110 via an RF link 185. Similarly, MDS 194 may be coupled to SCU 110 via another RF link 186. In a like manner, CPS 192 may be coupled to HHP 190 via an infra-red link 187; and MDS 194 may be coupled to HHP 190 via another infra-red link 188. Other types of telecommunicative links, other than RF or infra-red may also be used for this purpose. Through these links, CPS 192, for example, may be coupled through HHP 190 to SCU 110 for programming or diagnostic purposes. MDS 194 may also be coupled to SCU 110, either directly through RF link 186, or indirectly through the IR link 188, HHP 190, and RF link 185.

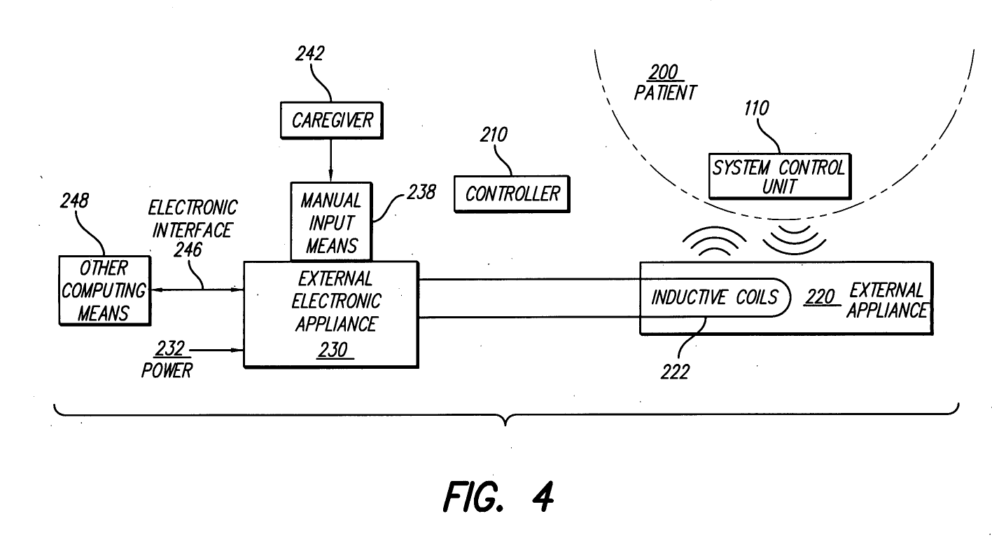

In another embodiment as illustrated in FIG. 4, the patient 200 switches SCU 110 on and off by use of controller 210, which is preferably hand held. Controller 210 operates to control SCU 110 by any of various means, including sensing the proximity of a permanent magnet located in controller 210, or sensing RF transmissions from controller 210.

External components for one preferred embodiment related to programming and providing power to SCU 110 are also illustrated in FIG. 4. When it is required to communicate with SCU 110, patient 200 is positioned on or near external appliance 220, which appliance contains one or more inductive coils 222 or other means of communicating with (e.g., RF transmitter and receiver), providing stimulation parameters to, or transferring power to a system control unit. External appliance 220 is connected to or is a part of external electronic circuitry appliance 230 which receives power 232 from a conventional power source. External appliance 230 contains manual input means 238, e.g., a keypad, whereby the patient 200 or a caregiver 242 may request changes in the parameters of the electrical and/or drug stimulation produced during the normal operation of SCU 110. In this preferred embodiment, manual input means 238 includes various electromechanical switches and visual display devices that provide the patient and/or care giver with information about the status and prior programming of SCU 110.

Alternatively or additionally, external electronic appliance 230 is provided with an electronic interface means 246 for interacting with other computing means 248, such as by a serial interface cable or infrared link to a personal computer or to a telephone modem. Such interface means 246 thus permits a clinician to monitor the status of the implant and prescribe new stimulation parameters from a remote location.

The external appliance(s) may advantageously be embedded in a cushion, pillow, or hat. Other possibilities exist, including a head band or other structure that may be affixed to the patient's body or clothing, such as a BTE unit worn behind the patient's ear.

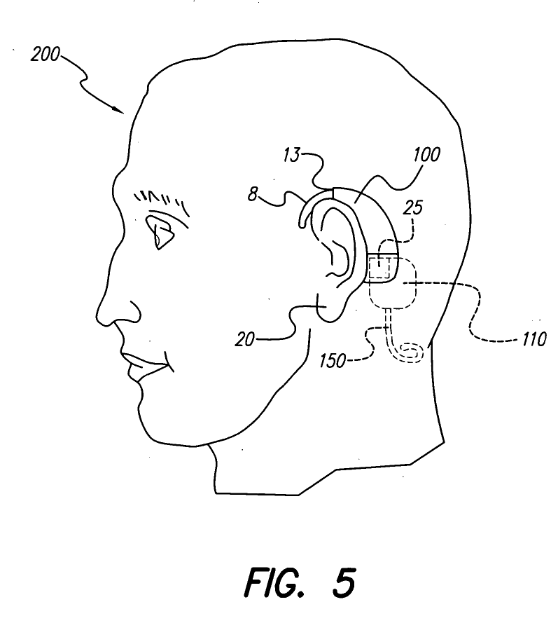

FIG. 5 shows, for example, a BTE unit 100 behind the ear of the patient 200. The BTE unit 100 may be attached 13 to an earhook 8 that secures the BTE unit 100 around the auricle of the ear. Additionally, the BTE unit 100 may include a magnet and/or metal plate 25 that is attracted to a corresponding magnet and/or metal plate in the SCU 110. This magnet and/or metal plates help secure the external BTE unit to the internal SCU 110 so that the two devices may maintain communication.

The BTE unit 100 is a preferred example of an external appliance 220 that includes electronic circuitry, a power source, and at least one RF coil, or other means of communicating with the SCU 110 as previously described. Purposes of the BTE unit 100 may include: providing power to the SCU 110; controlling, modifying, or monitoring the activities and/or parameters of the SCU 110; and/or providing a communications transfer to another external appliance, such as external appliance 230.

In the case where the SCU 110 is located in an area of the mastoid bone 143 such that communication between the SCU 110 and the BTE unit 100 is not practical or possible, the BTE unit 100 may alternately be in communication with a head piece that magnetically attracts to the SCU 110. The head piece includes all the components necessary to communicate with the SCU 110 in a manner that is either independent of or supported by the BTE unit 100. For example, the head piece includes at least an RF coil, or other means of communication, and related circuitry necessary to put the head piece in communication with the SCU 110.

In order to help determine the strength and/or duration of electrical stimulation required to produce the desired effect, in one preferred embodiment, a patient's response to and/or need for treatment is sensed. For example, the present invention may include an SCU that senses and measures the electrical activity of a neural population (e.g., EEG) or other relevant activities and substances that will be evident to those of skill in the art upon review of the present disclosure. The sensed and measured information is preferably used to control the stimulation parameters of the SCU(s) in a closed-loop manner.

While an SCU 110 may also incorporate means of sensing activity and/or substances, it may alternatively or additionally be desirable to use a separate or specialized implantable device to record and telemeter physiological conditions/responses in order to adjust electrical stimulation parameters. This information may be transmitted to an external device, such as external appliance 220, or may be transmitted directly to implanted SCU(s) 110. However, in some cases, it may not be necessary or desired to include a sensing function or device, in which case stimulation parameters are determined and refined, for instance, by patient feedback.

Thus, it is seen that in accordance with the present invention, one or more external appliances are preferably provided to interact with SCU 110 to accomplish one or more of the following functions:
Function 1: If necessary, transmit electrical power from the external electronic appliance 230 via appliance 220 to SCU 110 in order to power the device and/or recharge the power source/storage device 180. External electronic appliance 230 may include an automatic algorithm that adjusts electrical stimulation parameters automatically whenever the SCU(s) 110 is/are recharged.
Function 2: Transmit data from the external appliance 230 via the external appliance 220 to SCU 110 in order to change the parameters of electrical and/or drug stimulation produced by SCU 110.
Function 3: Transmit sensed data indicating a need for treatment or in response to stimulation from SCU 110 (e.g., impedance, electrical activity of a neural population (e.g., EEG), or other activity or substances) to external appliance 230 via external appliance 220.
Function 4: Transmit data indicating state of the SCU 110 (e.g., battery level, drug level, electrical stimulation and/or infusion settings, etc.) to external appliance 230 via external appliance 220.

By way of example, a treatment modality for a headache or neuralgia is carried out according to the following sequence of procedures:
1 An SCU 110 is implanted so that its electrodes 152 are located adjacent both branches of the greater occipital nerve 130 and both branches of the third occipital nerve 134. If necessary or desired, electrodes 152′ may additionally or alternatively be located in or near these or other adjacent nerves.
2 Using Function 2 described above (i.e., transmitting data) of external electronic appliance 230 and external appliance 220, SCU 110 is commanded to produce a series of excitatory electrical stimulation pulses, possibly with gradually increasing amplitude.
3. After each stimulation pulse, or at some other predefined interval, any change in electrical or other activity of a neural population (e.g., EEG) resulting from the electrical stimulation is sensed, preferably by one or more electrodes 152 and/or 152′. These responses are converted to data and telemetered out to external electronic appliance 230 via Function 3.
4. From the response data received at external appliance 230 from SCU 110, the stimulus threshold for obtaining a response is determined and is used by a clinician 242 acting directly 238 or by other computing means 248 to transmit the desired electrical parameters to SCU 110 in accordance with Function 2.
5. When patient 200 desires to invoke electrical stimulation, patient 200 employs controller 210 to set SCU 110 in a state where it delivers a clinician 242 prescribed stimulation pattern from a predetermined range of allowable stimulation patterns.
6. To cease electrical stimulation, patient 200 employs controller 210 to turn off SCU 110.
7. Periodically, the patient or caregiver recharges the power source/storage device 180 of SCU 110, if necessary, in accordance with Function 1 described above (i.e., transmit electrical power).

For the treatment of any of the various types and levels of headaches and/or neuralgias, it may be desirable to modify or adjust the algorithmic functions performed by the implanted and/or external components, as well as the surgical approaches, in ways that would be obvious and/or advantageous to skilled practitioners of these arts. For example, it may be desirable to employ more than one SCU 110, each of which could be separately controlled by means of a digital address. Multiple channels and/or multiple patterns of electrical and/or drug stimulation might thereby be programmed by the clinician and controlled by the patient in order to deal with complex or multiple symptoms or dysfunctions, such as a migraine headache with an occipital neuralgia.

In one embodiment, SCU 110, or a group of two or more SCUs, is controlled via closed-loop operation. A need for and/or response to stimulation is sensed via SCU 110, or by an additional SCU (which may or may not be dedicated to the sensing function), or by another implanted or external device. If necessary, the sensed information is transmitted to SCU 110. Preferably, the parameters used by SCU 110 are automatically adjusted based on the sensed information. Thus, the electrical and/or drug stimulation parameters are adjusted in a closed-loop manner to provide stimulation tailored to the need for and/or response to the electrical and/or drug stimulation.

In another embodiment, sensing means described earlier may be used to orchestrate first the activation of SCU(s) targeting one or more nerves of the upper cervical spine, and then, when appropriate, the SCU(s) targeting another area and/or by a different means. Alternatively, this orchestration may be programmed and not based on a sensed condition.

Thus, the present invention provides systems and methods for the treatment, control, and/or prevention of headaches and/or neuralgias of the upper cervical spine use at least one SCU. The present invention's advantages include, among others: monitoring and programming capabilities; power source, storage, and transfer mechanisms; device activation by the patient or clinician; open- and closed-loop capabilities coupled with sensing a need for and/or response to treatment; simple explantation because the IPG is implanted in the skull (e.g., the mastoid bone) and all leads are directly attached to the IPG; and coordinated use of one or more SCUs.

While the invention herein disclosed has been described by means of specific embodiments and applications thereof, numerous modifications and variations could be made thereto by those skilled in the art without departing from the scope of the invention set forth in the claims.

1. A method of treating patients with headaches and/or neuralgias, comprising:
implanting at least one system control unit in a shallow recess of the skull of a patient, wherein the at least one unit is capable of controlling the delivery of at least one stimulus to at least one nerve of the upper cervical spine; and
applying the at least one stimulus to the at least one nerve of the upper cervical spine of the patient being treated.

2. The method of claim 1 wherein the at least one nerve of the upper cervical spine is selected from at least one of the body, branches, and roots of at least one of the greater occipital nerves, the lesser occipital nerves, the third occipital nerves, the great auricular nerves, the transverse cervical nerves, and the supraclavicular nerves.

3. The method of claim 1 further comprising, implanting at least one system control unit in a shallow recess of the mastoid bone of the skull.

4. The method of claim 1 wherein the at least one system control unit is connected to at least one electrode, and wherein the stimulus comprises electrical stimulation deliverable via the at least one electrode.

5. The method of claim 1 further comprising sensing at least one condition and using the at least one sensed condition to automatically determine the stimulus to apply.

6. A system for treating patients with headaches and/or neuralgias, comprising:
at least one system control unit configured for implantation into a recess of the skull;
at least one stimulating electrode;
means for providing operating power to the at least one system control unit;
means for communicating with and providing stimulation parameters to the at least one system control unit; and
means for generating stimulation pulses in accordance with the stimulation parameters;
wherein the at least one electrode is configured to deliver the stimulation pulses to at least one nerve of the upper cervical spine.

7. The system of claim 6 wherein the system control unit is configured to conform to the profile of mastoid area of the skull.

8. The system of claim 6 wherein the means for providing operating power to the at least one system control unit and the means for communicating with and providing stimulation parameters to the at least one system control unit are include within a Behind-the-Ear unit.

9. A therapeutic system for patients with headaches and/or neuralgias, comprising:
at least one lead, wherein the at least one lead includes at least one electrode; and
at least one system control unit having a size and shape suitable for implantation in a recess in the skull, wherein the at least one system control unit comprises:
electronic circuitry that generates stimulation pulses in accordance with prescribed stimulation parameters, which electronic circuitry is operably connected to the at least one electrode through which the stimulation pulses may be delivered to tissue adjacent to the at least one electrode;
programmable memory for receiving and storing the prescribed stimulation parameters; and
a power source for providing operating power to the electronic circuitry.

10. The therapeutic system of claim 9 wherein the system control unit is configured to conform to the profile of the mastoid area of the skull.

11. The therapeutic system of claim 9 wherein the electronic circuitry is configured to generate stimulation pulses of at least 10 Hz.

12. The therapeutic system of claim 11 wherein the at least one electrode is configured to apply the stimulation pulses to at least one nerve of the upper cervical spine, wherein said at least one nerve is selected from at least one of the body, branches, and roots of at least one of the greater occipital nerves, the lesser occipital nerves, the third occipital nerves, the great auricular nerves, the transverse cervical nerves, and the supraclavicular nerves.

13. The therapeutic system of claim 9 wherein the system control unit further comprises at least one sensor.

14. The therapeutic system of claim 13 wherein the system control unit is configured to use the sensed condition to adjust the stimulation parameters.

15. The therapeutic system of claim 9 wherein the at least one lead includes at least one anchor.

16. The therapeutic system of claim 15 wherein the at least one anchor is a tine.

17. The therapeutic system of claim 9 wherein the system control unit further comprises electronic circuitry and means for communicating with an external appliance.

18. The therapeutic system of claim 17 wherein the external appliance is a Behind-the-Ear unit.

19. The therapeutic system of claim 18 wherein the Behind-the-Ear unit includes electronic circuitry and means for communicating with a second external appliance.
